(19) United States
(12) Patent Application Publication
(10) Pub. No.: US 2010/0286442 A1
(43) Pub. Date: Nov. 11, 2010

(54) NOVEL METHOD FOR PREPARING PREGABALIN

(75) Inventors: Hee-Seung Lee, Daejeon (KR); Joohee Lee, Incheon (KR); Taedong Ok, Seoul (KR)

Correspondence Address: SEED INTELLECTUAL PROPERTY LAW GROUP PLLC, 701 FIFTH AVE, SUITE 5400, SEATTLE, WA, 98104, US

(73) Assignee: KAIST, Daejeon (KR)

(21) Appl. No.: 12/677,494

(22) PCT Filed: Aug. 11, 2008
(86) PCT No.: PCT/KR2008/004672

§ 371 Date: Jul. 14, 2010

(30) Foreign Application Priority Data
Aug. 10, 2007 (KR) 10-2007-0080922

(51) Int. Cl. C07C 229/08

(52) U.S. Cl. 562/553

(57) ABSTRACT

The present invention relates to a method for preparing pregabalin ((S)-3-(aminomethyl)-5-methylhexanoic acid which is useful for the prevention and treatment of seizure disorders, pins, and psychiatric disorders. According to the present invention, pregabalin can be prepared in a high enantiomeric excess of 99% or more, without an additional step of separating or purifying its enantiomer.

### TECHNICAL FIELD

The present invention relates to a method for preparing (S)-3-(aminomethyl)-5-methylhexanoic acid of the following Formula 1, which is widely known as an anticonvulsant for treating and preventing neuropathic pain.

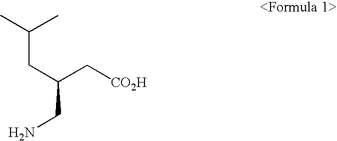

### BACKGROUND ART

(S)-(+)-3-(aminomethyl)-5-methylhexanoic acid is generally known as (S)-pregabalin, and also called (S)-(+)-β-isobutyl-γ-aminobutyric acid, (S)-3-isobutyl-GABA, or CI-1008. (S)-Pregabalin, marketed under the trade name LYRICA, is a neurotransmitter modulator that is effective for the treatment of neuropathic pain, seizure and generalized anxiety disorder, and is known to have a more rapid onset of action and be convenient to use. Thus, it is known to significantly alleviate a patient's symptoms, compared with other therapeutic agents for each disease (U.S. Pat. No. 5,563,175).

It was reported that chronic pain syndrome is associated with excessive neuronal activity and can be treated by reducing the concentration of neurotransmitters. Pregabalin, gabapentinoid drug, has a unique mechanism of action which allows treatment of certain neurologic and psychiatric disorders. Pregabalin modulates the voltage-dependent calcium channel in the central nervous system to increase the concentration of an endogenous inhibitory neurotransmitter, γ-aminobutyric acid or GABA (gamma-aminobutyric acid), resulting in the treatment of certain neurologic disorders, pains, and psychiatric disorders (Nature Reviews Drug Discovery 2005, 4, 455).

The anticonvulsant effect of racemic isobutyl-GABA is primarily attributable to the (S)-enantiomer, pregabalin (Bioorg. Med. Chem. Lett., 1994, 4, 823). Thus, the commercial utility of pregabalin requires an efficient method for preparing the (S)-enantiomer with a high enantiomeric excess (hereinafter, referred to as “ee”).

Typically, a racemic mixture of 3-(aminomethyl)-5-methyl-hexanoic acid is synthesized and subsequently resolved into its (R)- and (S)-enantiomers. Such methods may employ an azide intermediate (Richard Silverman et al., Synthesis, 1989, 953., U.S. Pat. No. 5,563,175), a malonate intermediate (Grote et al., U.S. Pat. Nos. 6,046,353, 5,840,956, and 5,637,767), or Hofmann synthesis (Huckabee and Sobieray, U.S. Pat. Nos. 5,629,447 and 5,616,793). In these methods, the classical method of resolving a racemate is used to separate and purify the desired (S)-enantiomer. Classical resolution involves preparation of a salt with a chiral resolving agent to separate and purify the desired (S)-enantiomer, and also substantial additional cost associated with the resolving agent. Partial recycling of the resolving agent is feasible, but this is associated with waste generation. Moreover, the maximum theoretical yield of pregabalin is 50%, since only half of the racemate is the desired product and the undesired (R)-enantiomer is ultimately discarded as waste. This reduces the effective throughput of the process (the amount that can be made in a given reactor volume) by 50% or less.

Pregabalin has been also synthesized by stereoselective synthesis using chiral auxiliary, (4R,5S)-4-methyl-5-phenyl-2-oxazolidinone (Richard Silverman et al., U.S. Pat. Nos. 6,359,169, 6,028,214, 5,847,151, 5,710,304, 5,684,189, 5,608,090 and 5,599,973). Although these methods provide pregabalin in high enantiomeric purity, they are not practical for large-scale synthesis because they employ costly reagents which are difficult to handle, as well as special cryogenic equipment to reach the required operating temperatures.

Pregabalin can be also synthesized by asymmetric reaction using a catalyst. In this regard, US Patent Application No. 2003/0212290 describes a method of making pregabalin using a chiral rhodium catalyst via asymmetric hydrogenation of a cyano-substituted olefin to produce a chiral cyano precursor of (S)-3-(aminomethyl)-5-methylhexanoic acid. The cyano precursor is subsequently reduced to yield pregabalin. However, the method may create serious safety problems in large scale synthesis, because of using high levels of carbon monoxide gas in the preparation of the starting material, cyano-substituted olefin. In addition, pregabalin can be also synthesized by asymmetric cyanation using an Al-(Salen) catalyst (Jacobsen et al., J. Am. Chem. Soc. 2003, 125, 4442). However, the method is also not practical for large-scale synthesis, since its enantiomeric excess is as low as 96% ee and toxic reagents such as HCN and high-pressure hydrogen (500 psi) treatment are needed.

### DISCLOSURE

### Technical Problem

There is a need to develop a novel method for preparing pregabalin for low-cost, large-scale synthesis, which offers several advantages over previous methods in terms of yield and enantiomeric excess.

### Technical Solution

Accordingly, it is an object of the present invention to provide a novel method for preparing pregabalin, the (S) enantiomer of (S)-3-(aminomethyl)-5-methylhexanoic acid with a high enantiomeric excess.

### ADVANTAGEOUS EFFECTS

According to the novel method for preparing pregabalin of the present invention, pregabalin can be simply prepared from chiral bicyclic lactone with a high enantiomeric excess and yield, including the yield at each step of 70% or more and the overall yield of 50% or more.

According to the present invention, pregabalin can be also prepared in a high enantiomeric excess (ee) of 99% or more without a resolution step that is required in the conventional method involving an equivalent weight of a chiral auxiliary or a classical resolution method. In little time, only the desired (S)-enantiomer is obtained, without an additional step of removing the undesired (R)-enantiomer.

In addition, pregabalin, useful for the prevention and treatment of certain seizure disorders, pains, and psychiatric disorders, can be easily prepared according to the present invention, compared to the conventional methods involving materials such as a hazardous nitro compound, costly chiral auxiliaries and high-pressure gas, or cryogenic conditions.

### DESCRIPTION OF DRAWINGS

FIG. 1 shows the overall reaction process for preparing pregabalin according to the present invention;

FIG. 2 is the result of chiral GC analysis of the compound prepared in Example 1;

FIG. 3 is a ¹H NMR spectrum of the compound prepared in Example 1;

FIG. 4 is a ¹³C NMR spectrum of the compound prepared in Example 1;

FIG. 5 is a ¹H NMR spectrum of the compound prepared in Example 2;

FIG. 6 is a ¹³C NMR spectrum of the compound prepared in Example 2;

FIG. 7 is a ¹H NMR spectrum of Compound 3;

FIG. 8 is a ¹³C NMR spectrum of Compound 3;

FIG. 9 is a ¹H NMR spectrum of the compound prepared in Example 4;

FIG. 10 is a ¹³C NMR spectrum of the compound prepared in Example 4;

FIG. 11 is a ¹H NMR spectrum of the compound prepared in Example 5;

FIG. 12 is a ¹³C NMR spectrum of the compound prepared in Example 5;

FIG. 13 is the result of chiral GC analysis of the compound prepared in Example 5;

FIG. 14 is a ¹H NMR spectrum of Compound 4;

FIG. 15 is a ¹³C NMR spectrum of Compound 4;

FIG. 16 is a ¹H NMR spectrum of Compound 1; and

FIG. 17 is a ¹³C NMR spectrum of Compound 1.

### BEST MODE

With respect to the objects of the present invention, as shown in the following <Reaction Scheme 1>, the present invention provides a method for preparing pregabalin of Formula 1, comprising the steps of:

1) preparing a lactone compound (Compound 3) of the following Formula 3 via cyclopropane ring-opening reaction and decarboxylation of a bicyclic lactone compound (Compound 2) of the following Formula 2 by nucleophilic addition of isopropylcuprate;

2) preparing a compound (Compound 4) of the following Formula 4 via sequential reactions of halogenation, azidation, and hydrolysis of the lactone compound (Compound 3) of the following Formula 3 by lactone ring-opening reaction; and

3) preparing pregabalin (compound 1) of the following Formula 1 by reduction of the compound (compound 4) of the following Formula 4:

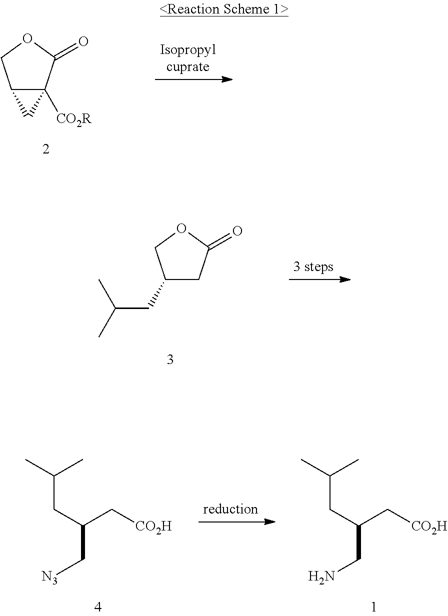

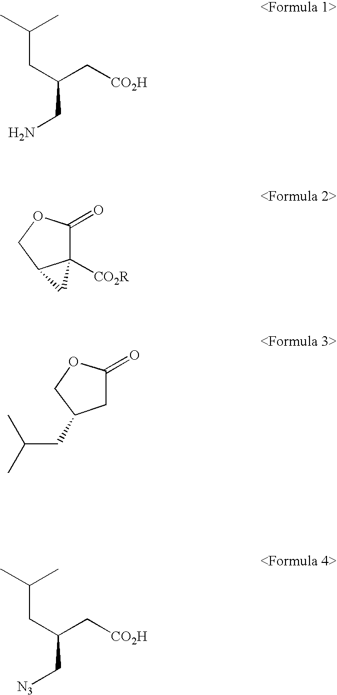

In Reaction Scheme 1 and Formula 2, R is a straight or branched hydrocarbon group having 1 to 6 carbon atoms, exemplified by alkyl such as methyl, ethyl, n-propyl, isopropyl, n-butyl, sec-butyl, isobutyl, tert-butyl, n-pentyl and n-hexyl, preferably lower alkyl including methyl, ethyl, n-propyl, isopropyl, and tert-butyl.

Hereinafter, the present invention will be described in detail.

Unlike the known methods, the method of the present invention is characterized in that the method comprises the step of preparing a β-substituted γ-butyrolactone intermediate of Formula 3 via cyclopropane ring-opening reaction of bicyclic lactone. In addition, the method of the present invention is characterized in that bicyclic lactone of Formula 2 having a high enantiomeric excess is used as a starting material to prepare pregabalin that is an enantiomer having (S) configuration at β-carbon of 3-(aminomethyl)-5-methylhexanoic acid.

As shown in the following Reaction Scheme 2, in step 1) of the present invention, nucleophilic addition of isopropylcuprate, prepared in-situ in a reaction solution containing isopropylmagnesium halide represented by i-PrMgX and a copper compound represented by CuY, to the bicyclic lactone compound of Formula 2 (Compound 2) is performed to prepare the compound of Formula 5 (Compound 5), followed by decarboxylation to prepare the compound of Formula 3 (Compound 3) in a yield of 70% or more:

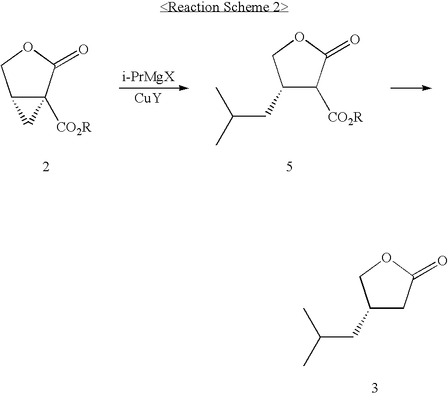

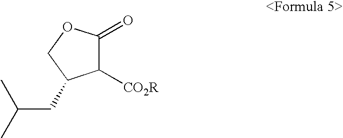

In Formula 5, R is the same as defined in Formula 2.

The starting material, bicyclic lactone compound represented by Formula 2 (Compound 2) preferably has an enantiomeric excess of 99% ee or higher.

In Formula i-PrMgX, which represents isopropylmagnesium halide used in the nucleophilic addition of Reaction Scheme 2, i-Pr may be an isopropyl group, and X may be any one of Cl, Br, and I, preferably Cl. In Formula CuY, which represents the copper compound, Y may be any one of Cl, Br, I, and CN, preferably I.

Examples of the solvent used for the nucleophilic addition may include anhydrous solvents such diethyl ether, tetrahydrofuran, hexane, and heptane, and these solvents may be used alone or in combination of two or more thereof. The reaction temperature may vary depending on the used solvent, ranging from −50 to 0° C. preferably −50 to −40° C. The reaction time may also vary depending on the reaction temperature and the used solvent, ranging from 1 to 18 hrs. In addition, based on the bicyclic lactone compound of Formula 2, the copper compound (CuY) and isopropylmagnesium halide (i-PrMgX) are preferably used in an amount of 0.05 to 0.95 equivalent weight and 1.1 to 10 weight equivalents, respectively.

The decarboxylation may be accomplished by heating a reactor under typical decarboxylation conditions, and preferably performed by heating the reactor at 100 to 150° C. more preferably performed in a mixed solvent of LiCl/water/DMSO.

As shown in the following Reaction Scheme 3, in step 2) of the present invention, the compound of Formula 3 (Compound 3) obtained in step 1) sequentially undergoes three steps of (a) halogenation, (b) azidation, and (c) hydrolysis to give the compound of Formula 4 (Compound 4). Briefly, in step 2), the compound of Formula 3 (Compound 3) is converted into the compound of Formula 4 (Compound 4) via a compound of the following Formula 6 (Compound 6) and a compound of the following Formula 7 (Compound 7).

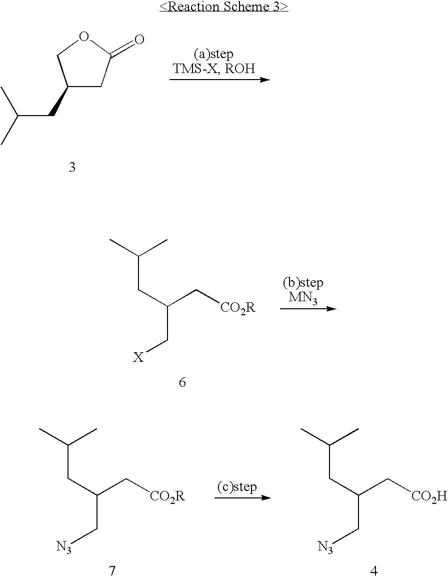

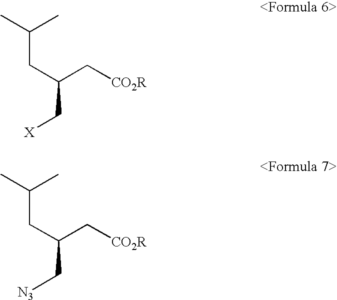

In TMS-X of Reaction Scheme 3, which represents trimethylsilyl halide, TMS is a trimethylsilyl group ((CH₃)₃Si—), and X is halide including Br and I, preferably Br. In Formula ROH representing alcohol, R may be alkyl or aryl, and the alcohol is preferably ethanol. MN₃ represents an azide compound, in which M may be a compound of Group IA including Na and K, and preferably Na.

In Formulae 6 and 7, R is the same as defined in Formula 2.

Each step of three reactions included in step 2) of the present invention will be described with reference to Reaction Scheme 3, as follows.

(a) Halogenation

In step (a) of the present invention, the compound of Formula 3 obtained in step 1) is reacted with alcohol represented by ROH and trimethylsilyl halide (TMS-X) to form the halogen compound of Formula 6.

Halide of trimethylsilyl halide is substituted into the γ position of the lactone ring of the compound of Formula 3 (Compound 3) to open the lactone ring, and its yield is 90% or more. In this regard, based on the compound of Formula 3, alcohol and trimethylsilyl halide are preferably used in an amount of 1 to 10 weight equivalents and 1 to 10 weight equivalents, respectively.

(b) Azidation

In step (b) of the present invention, the halogen compound of Formula 6 (Compound 6) obtained in step (a) is reacted with an azide compound (MN₃) to obtain the compound of Formula 7 (Compound 7). In this regard, the azide compound is preferably used in an amount of 1 to 10 weight equivalents, based on the compound of Formula 6.

(c) Hydrolysis

In step (c) of the present invention, the compound of Formula 7 (Compound 7) obtained in step (b) is subjected to hydrolysis in a suitable solvent in the presence of a base, so as to obtain the azide compound of Formula 4 (Compound 4). In this regard, examples of the solvent include alcohol such as methanol and ethanol and/or aqueous solvents such as tetrahydrofuran (THF) miscible with water, and preferably a mixed solvent of THF, methanol, and water. The base may vary depending on an alkali salt of carboxylic acid, and include alkali metal hydroxide such as lithium hydroxide, sodium hydroxide, and potassium hydroxide, preferably lithium hydroxide.

To obtain the compound of Formula 4 (Compound 4) in a form of carboxylic acid, acetic acid or 1 to 6 N hydrochloric acid aqueous solutions may be added.

In step 3) of the present invention, an azide functional group of the azide compound of Formula 4 obtained in step 2) is reduced to an amine group, so as to obtain a target material, pregabalin. The reduction may be performed by various known methods, and preferably performed by using a palladium-carbon (Pd/C) catalyst in a suitable solvent such as methanol. In this regard, when the chiral GC analysis is performed using a chiral DEX β-DM column (130° C. 1.41 kgf/cd), pregabalin of Formula 1 has an enantiomeric excess of 99% or more.

The present invention is characterized in that the bicyclic lactone compound of Formula 2 is used as a starting material to prepare pregabalin via the β-substituted γ-butyrolactone intermediate. The bicyclic lactone compound of Formula 2 may be prepared by the method that is described in {crystalline forms of bicyclic lactone and preparation method thereof} applied by the present inventors. According to the method, the compound of Formula 2, as shown in the following Reaction Scheme 4, is reacted with (S)-epichlorohydrin of Formula 8 and malonate of Formula 9 (Compound 9), and then the reaction mixture is distilled under vacuum to remove the solvent and unreacted dialkylmalonate, followed by cooling to obtain a pure crystalline form having an enantiomeric excess of 99% or more:

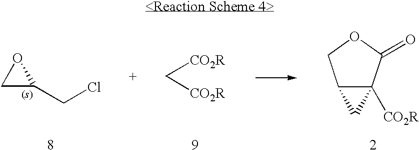

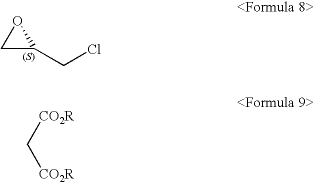

In Reaction Scheme 4 and Formula 9, R is the same as defined in Formula 2.

In this connection, the malonate of Formula 9 (Compound 9) is preferably diethylmalonate.

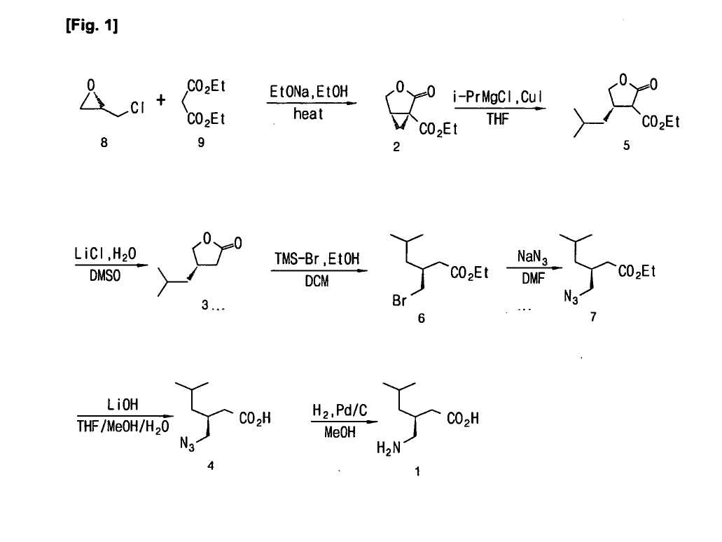

Thus, according to the preparation method of the present invention, bicyclic lactone having a high enantiomeric excess can be used to prepare pregabalin having a high enantiomeric excess via the β-substituted γ-butyrolactone intermediate. The overall reaction process for preparing pregabalin according to the present invention is illustrated in FIG. 1.

Various publications are cited herein which are hereby incorporated, by reference, in their entireties. The present invention is not to be limited in scope by the embodiments disclosed in the examples which are intended as an illustration of one aspect of the invention, and any compositions or methods which are functionally equivalent are within the scope of this invention.

Indeed, various modifications or variations of the invention in addition to those shown and described herein will become apparent to those skilled in the art from the foregoing description. Such modifications and variations are intended to fall within the scope of the present invention. Additionally, various exemplary components, compositions, properties and steps described herein may be used alone or in any combination of two or more thereof.

### MODE FOR INVENTION

Hereinafter, the preparation method of pregabalin and intermediate compounds obtained during the process will be described with reference to Examples. The present invention may, however, be embodied in many different forms and should not be construed as being limited to the Examples set forth herein.

### Example 1

### Preparation of (1R,5S)-ethyl 2-oxo-3-oxa-bicyclo[3.1.0]hexane-1-carboxylate

Sodium pieces (3.09 g, 134 mmol) were added to anhydrous ethanol (250 mL), and stirred for 30 min until completely dissolved to obtain an ethoxide solution. The ethoxide solution was cooled to 0° C. and then 21.4 mL (141.0 mmol) of diethylmalonate was slowly added dropwise. Then, the temperature was increased to room temperature, and 10 mL (127.9 mmol) of (S)-epichlorohydrin was slowly added dropwise using a syringe pump, the reaction mixture was heated at 75° C. for 36 hrs. Distilled water was added to the reaction mixture until the solution became clear. When the reaction mixture became clear, ethanol was removed therefrom under reduced pressure. An aqueous layer was extracted with methylene chloride, and then the organic layer was dried over anhydrous magnesium sulfate, and the residue was concentrated under reduced pressure. The concentrate was distilled under vacuum at 1.5 mmHg, and the produced oil was refined according to the following procedure to give the title compound ((1R,5S)-ethyl 2-oxo-3-oxa-bicyclo[3.1.0]hexane-1-carboxylate).

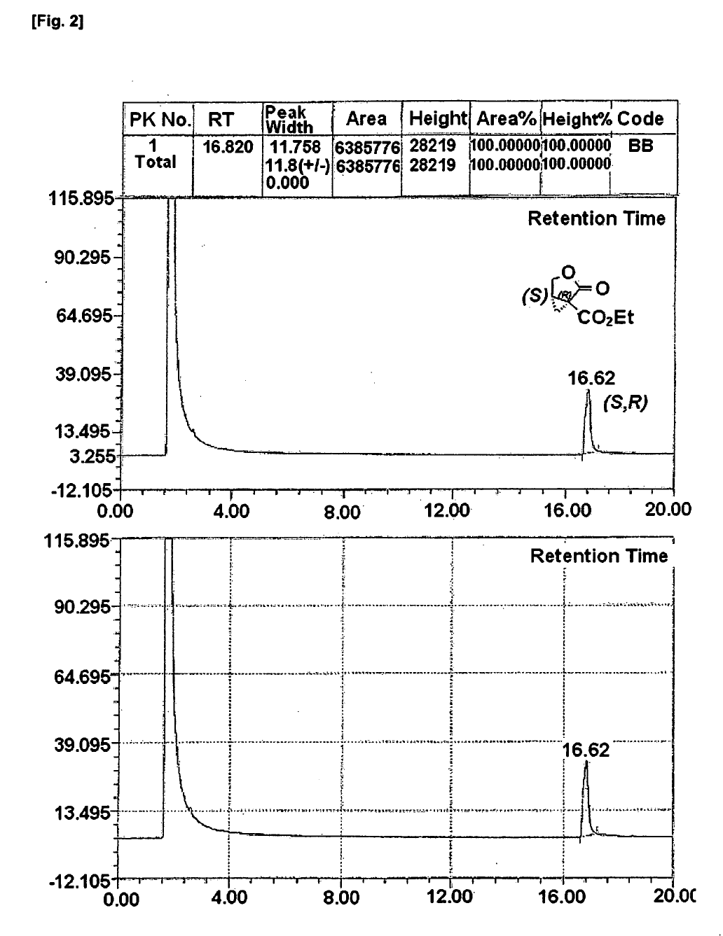

The unreacted diethylmalonate was removed at 42 to 43° C. and then the title compound was obtained at 110 to 112° C. as a clear oil (12.23 g, 59%). The oil was stored at −20° C. and the title compound of Example 1 was solidified in a needle-like crystalline. Its enantiomeric excess (ee) was 99% or more, when chiral GC analysis was performed using a chiral DEX β-DM column (130° C. 1.41 kgf/cm¹ tᵣ=16.88), as shown in FIG. 2. In this regard, [α]²⁰_{D} was +166.39 (c 1.22, EtOH), and [α]²⁵_{D}, was +134.81 (c 1.00, CH₂Cl₂) (literature value: [α]²⁵_{D}=+145.48 (c 1.22, EtOH) for >97% ee).

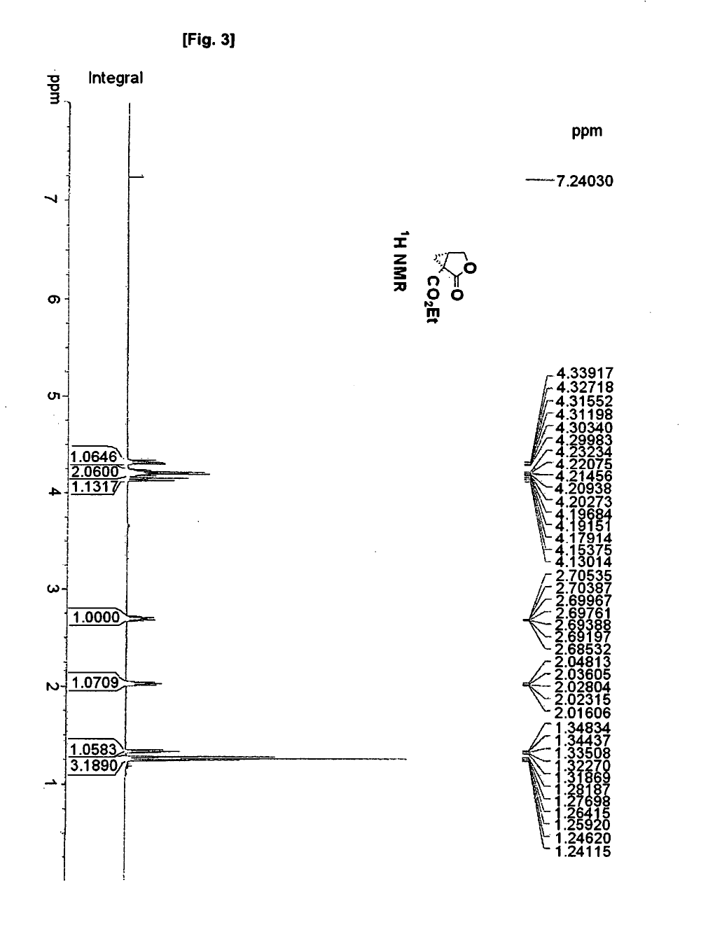

The result of ¹H NMR (400 MHz, CDCl₃) analysis of the title compound was as follows: δ 4.33 (1H, dd, J=4.73 Hz and 9.42 Hz), 4.23 (2H, q, J=7.14 Hz), 4.16 (1H, d, J=9.43 Hz), 2.70 (1H, m), 2.05 (1H, dd, J=4.77 Hz and 7.98 Hz), 1.35 (1H, t, J=5.14 Hz), 1.28 (3H, t, J=7.13 Hz), shown in FIG. 3.

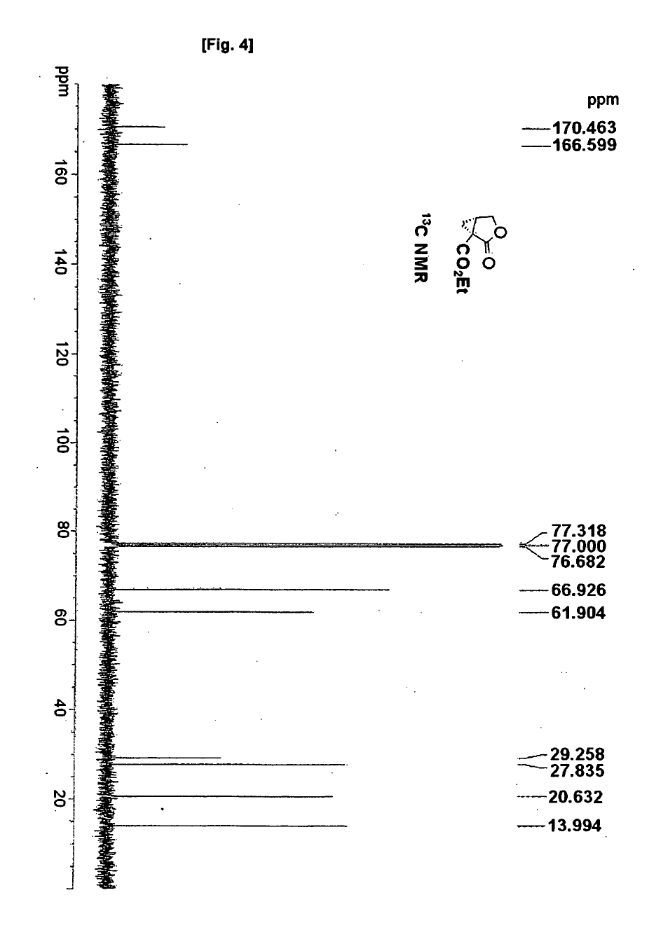

The result of ¹³C NMR (100 MHz, CDCl₃) analysis 5 of the title compound was as follows: δ 170.5, 166.7, 67.0, 62.0, 29.3, 27.9, 20.7, 14.1, shown in FIG. 4.

The result of HRMS (EI)(C₈H₁₀O₄) was as follows: calculated value=170.0579, measured value=170.0571.

### Example 2

### Preparation of (4S)-ethyl tetrahydro-4-isobutyl-2-oxofuran-3-carboxylate

CuI (0.63 g, 3.31 mmol) was added to anhydrous THF (20 mL) at −45° C. and the suspension was stirred. Then, isopropylmagnesium chloride (THF solvent, 2.0M, 8.23 mL, 16.46 mmol) was slowly added dropwise to the stirred suspension. The ((1R,5S)-ethyl 2-oxo-3-oxa-bicyclo[3.1.0]hexane-1-carboxylate, 1.12 g (6.58 mmol) prepared in Example 1 that was dissolved in anhydrous THF (20 mL) was added to the suspension using a cannular at −45° C. The reaction mixture was slowly stirred for 30 min to −15° C. and quenched with a saturated ammonium chloride solution. A suitable amount of diethyl ether was added thereto at room temperature, followed by stirring for 7-8 hrs. The ether layer was separated, and the aqueous layer was extracted with ethyl acetate twice. Two organic layers were combined, and then dried over anhydrous magnesium sulfate, concentrated under reduced pressure. The residue was purified by column chromatography using a silica gel (10% ethylacetate/hexane) to obtain the title compound ((4S)-ethyl tetrahydro-4-isobutyl-2-oxofuran-3-carboxylate) as a colorless oil (1.34 g, 95%).

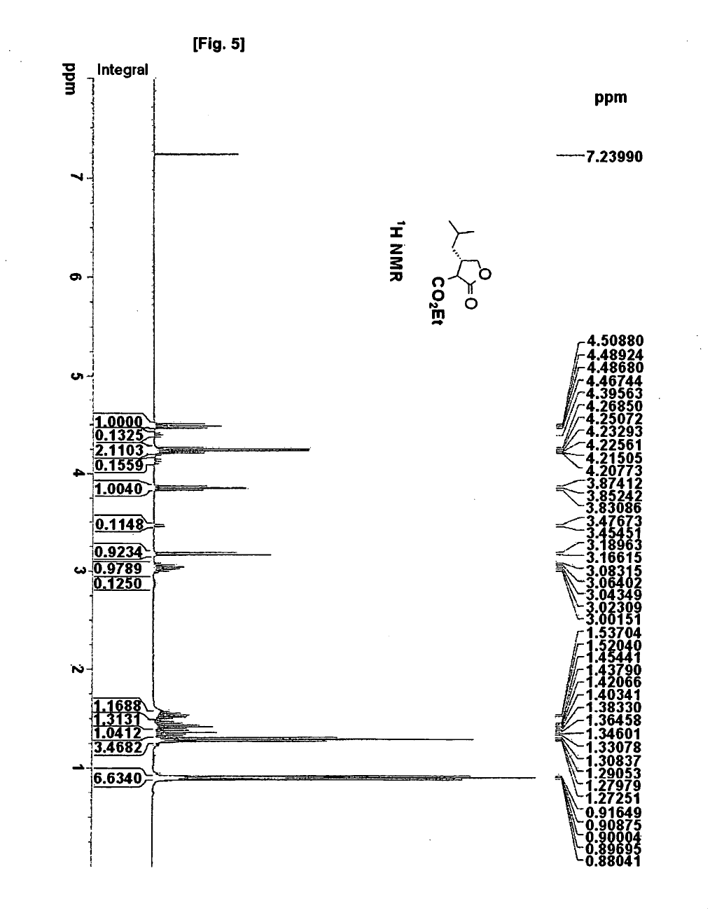

The result of H¹ NMR (400 MHz, CDCl₃) of the unrefined title compound was as follows: δ 4.49 (1H, dd, J=8.80 Hz and 7.82 Hz), 4.23 (2H, q, J=7.11 Hz), 3.85 (1H, t, J=8.68 Hz), 3.18 (1H, d, J=9.39 Hz), 3.03 (1H, m), 1.53 (1H, m), 1.42 (1H, m), 1.38 (1H, m), 1.31 (3H, t, J=8.96 Hz), 0.90 (6H, t, J=6.62 Hz), shown in FIG. 5.

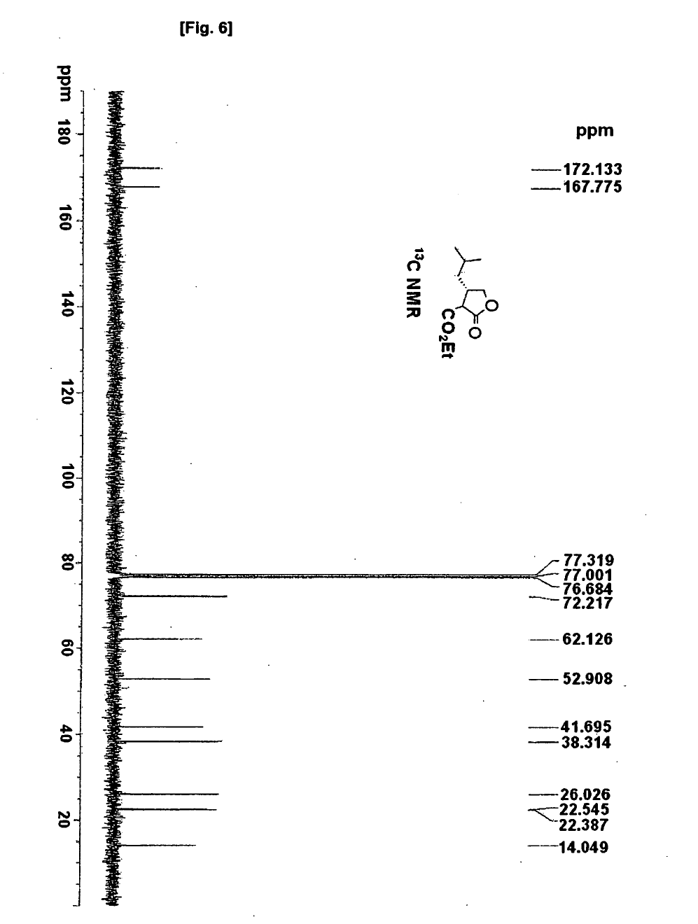

The result of ¹³C NMR (100 MHz, CDCl₃) of the unrefined title compound was as follows: δ 172.1, 167.8, 72.2, 62.1, 52.9, 41.7, 38.3, 26.0, 22.6, 22.4, 14.1, shown in FIG. 6.

The result of HRMS (EI) (C₁₁H₁₃O₄) was as follows: calculated value=214.1205, measured value=214.1208.

### Example 3

### Preparation of (S)-dihydro-4-isobutylfuran-2(3H)-one (Compound 3)

The compound ((4S)-ethyl tetrahydro-4-isobutyl-2-oxofuran-3-carboxylate, 991 mg, 4.63 mmol) prepared in Example 2 and LiCl (392 mg, 9.25 mmol) were dissolved in DMSO (50 mL), and then 1 mL of distilled water was added thereto, followed by stirring at 140° C. for 18 hrs. After the reaction was completed, water was added, and the mixture was extracted with ethyl acetate three times. The organic layer was washed with a saturated ammonium chloride solution and brine once, respectively. The organic layer was dried over anhydrous sodium sulfate, and concentrated under reduced pressure. The residue was purified by column chromatography using a silica gel (10% ethylacetate/hexane) to obtain Compound 3 ((S)-dihydro-4-isobutylfuran-2(3H)-one) as a colorless oil (520 mg, 79.1%).

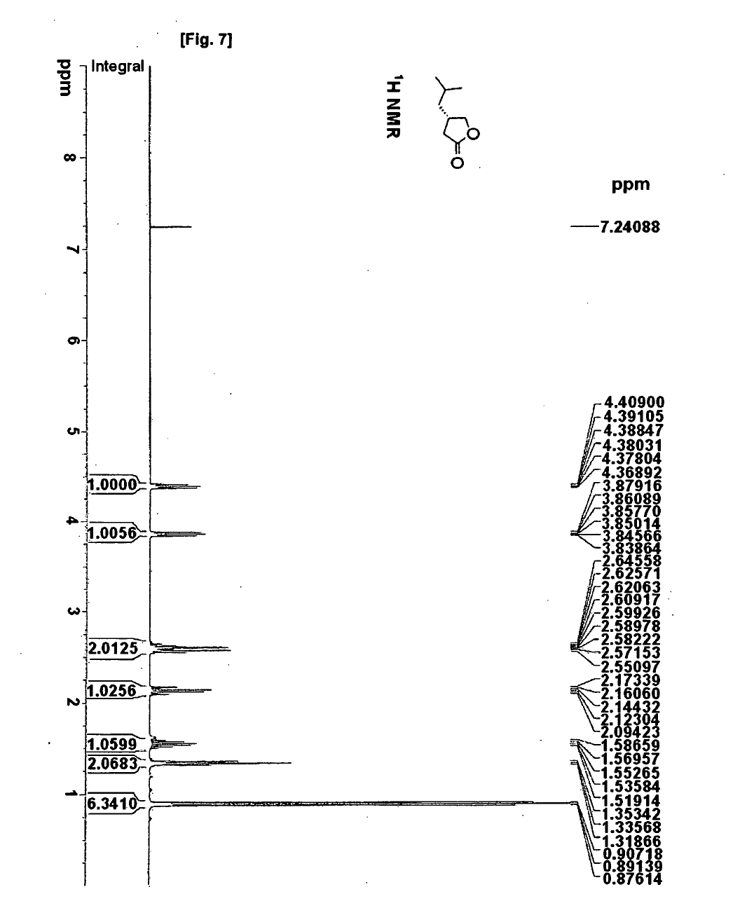

The result of H¹ NMR (400 MHz, CDCl₃) of Compound 3 was as follows: δ 4.39 (1H, t, J=8.53 Hz), 3.86 (1H, t, J=8.74 Hz), 2.60 (2H, m), 2.13 (1H, m), 1.55 (1H, m), 1.34 (2H, t, J=6.95 Hz), 0.89 (6H, t, J=6.21 Hz), shown in FIG. 7.

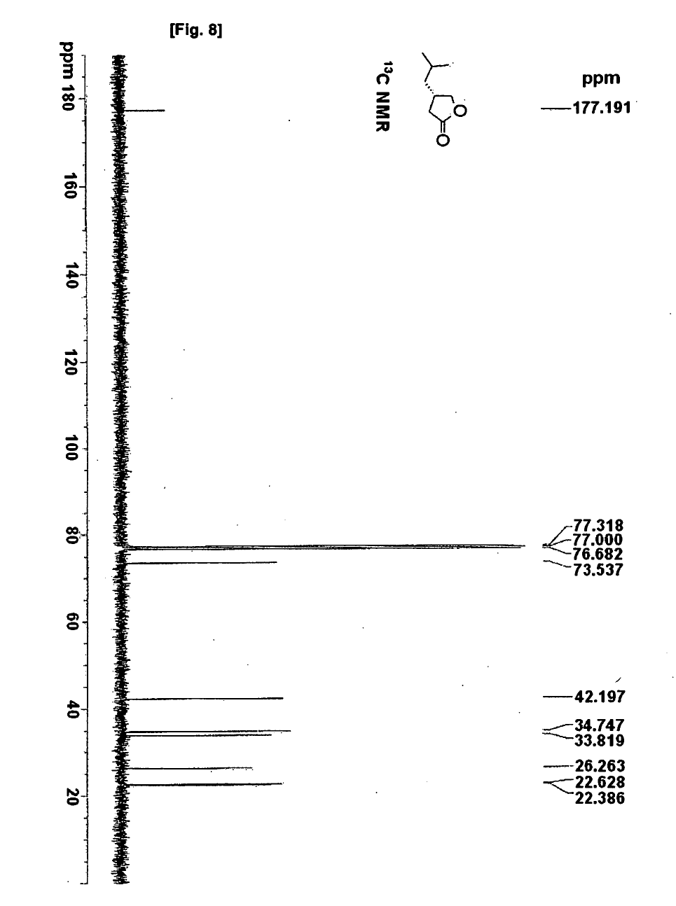

The result of ¹³C NMR (100 MHz, CDCl₃) was as follows: δ 177.2, 73.5, 42.2, 34.8, 33.8, 26.3, 22.6, 22.4, shown in FIG. 8.

The result of HRMS (EI) C₁₈H₁₄O₂) was as follows: calculated value=142.0994, measured value=142.0990.

### Example 4

### Preparation of (S)-Ethyl 3-(bromomethyl)-5-methylhexanoate

Compound 3 (1.11 g, 7.81 mmol) prepared in Example 3 and ethanol (2.28 mL, 39.0 mmol) were mixed with anhydrous methylene chloride (40 mL), and stirred. Bromotrimethylsilane (3.03 mL, 23.4 mmol) was slowly added dropwise to the stirred mixture at 0° C. The reaction mixture was stirred at room temperature for 18 hrs, and then quenched with distilled water, followed by stirring for 5 min. The organic layer was separated, and then washed with a 5% sodium thiosulfate solution. Then, the organic layer was dried over anhydrous sodium sulfate, and concentrated under reduced pressure. The residue was purified by column chromatography using a silica gel (5% ethylacetate/hexane) to obtain the title compound ((S)-ethyl 3-(bromomethyl)-5-methylhexanoate) as a colorless oil (1.77 g, 90.3%).

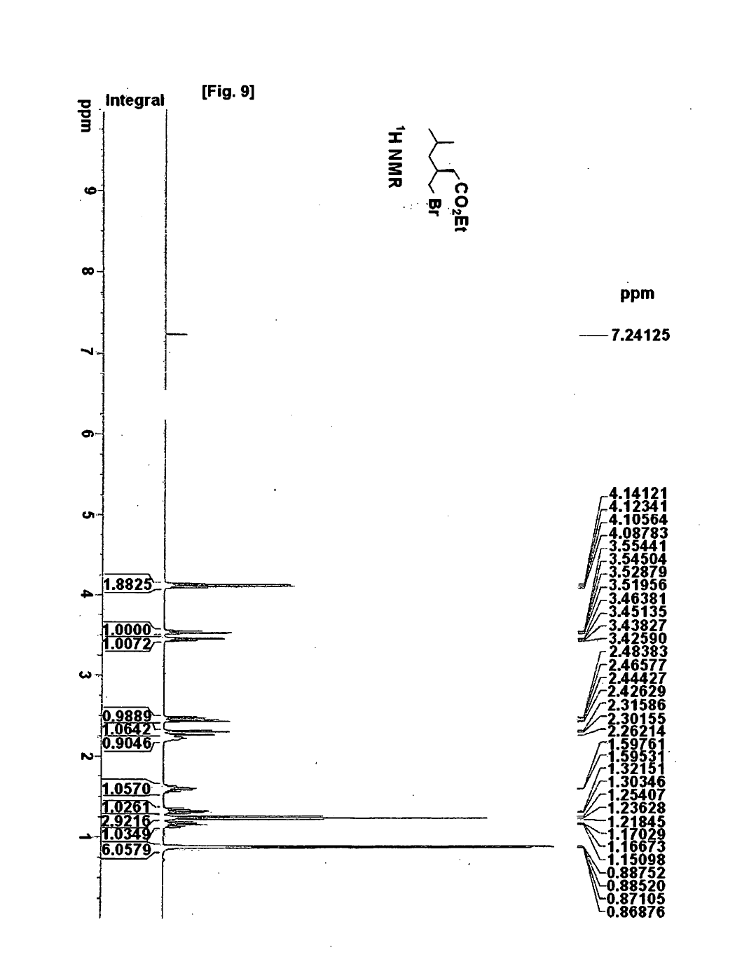

The result of H¹ NMR (400 MHz, CDCl₃) of the title compound was as follows: δ 4.11 (2H, q, J=7.12 Hz), 3.54 (1H, dd, J=10.22 Hz and 3.72 Hz), 3.44 (1H, dd, J=10.20 Hz and 4.97 Hz), 2.45 (1H, dd, J=15.81 Hz and 7.19 Hz), 2.29 (1H, dd, J=15.76 Hz and 5.72 Hz), 2.22 (1H, m), 1.60 (1H, m), 1.32 (1H, m), 1.24 (3H, t, J=7.12 Hz), 1.15 (1H, m), 0.88 (6H, d, J=6.58 Hz), shown in FIG. 9.

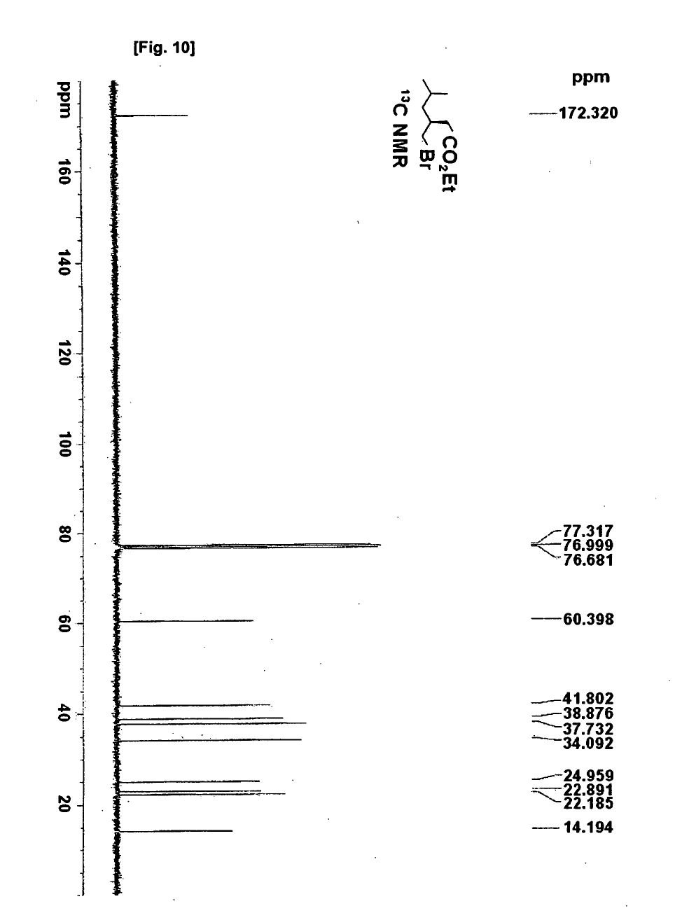

The result of ¹³C NMR (100 MHz, CDCl₃) of the title compound was as follows: δ 172.3, 60.4, 41.8, 38.98, 37.7, 34.1, 25.0, 22.9, 22.2, 14.2, shown in FIG. 10.

The result of HRMS (EI) (C₁₀H₁₉BrO₂) was as follows: calculated value=250.0568, measured value=250.0565.

### Example 5

### Preparation of (S)-ethyl 3-(azidomethyl)-5-methylhexanoate

The compound ((S)-ethyl 3-(bromomethyl)-5-methylhexanoate, 876 mg, 3.49 mmol) prepared in Example 4 and sodium azide (907 mg, 13.95 mmol) were mixed with anhydrous DMF (10 mL), and then stirred at room temperature for 4 hrs. DMF was removed therefrom under reduced pressure. Distilled water and methylene chloride were added to the residue. The aqueous layer was extracted with methylene chloride three times, and dried over anhydrous sodium sulfate, followed by concentration under reduced pressure. The concentrated residue was purified by column chromatography using a silica gel (5% ethylacetate/hexane) to obtain the title compound ((S)-ethyl 3-(azidomethyl)-5-methylhexanoate) as a colorless oil (691 mg, 92.9%).

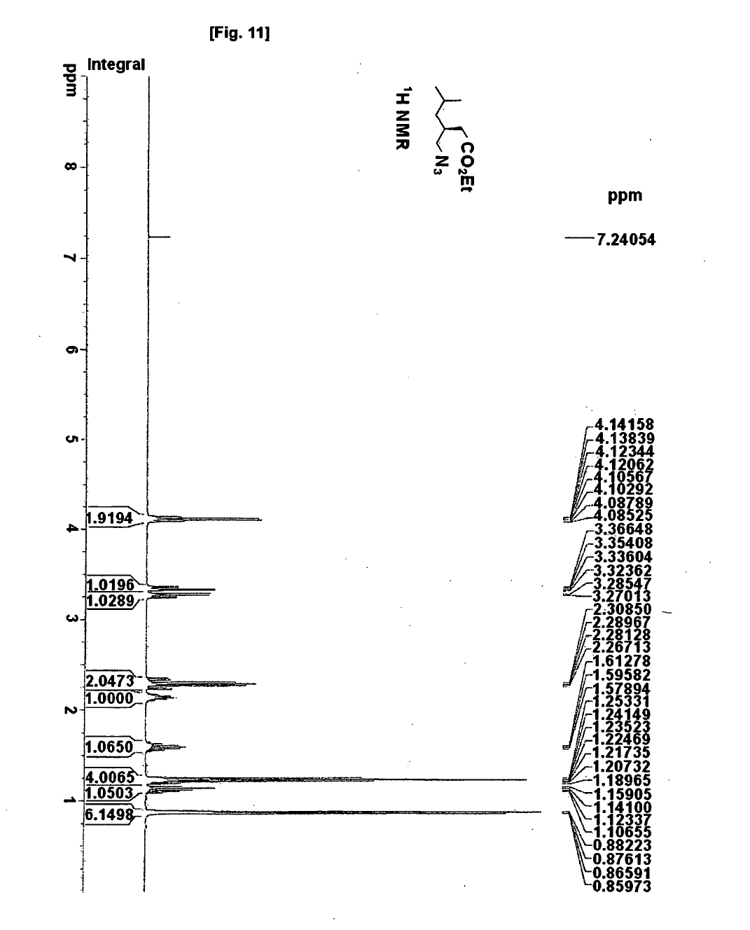

The result of H¹ NMR (400 MHz, CDCl₃) of the title compound was as follows: δ 4.11 (2H, q, J=7.16 Hz), 3.34 (1H, dd, J=12.18 Hz and 4.97 Hz), 3.26 (1H, dd, J=12.12 Hz and 6.14 Hz), 2.29 (2H, m), 2.14 (1H, m), 1.60 (1H, m), 1.24 (3H+1H, m), 1.13 (1H, m), 0.87 (6H, dd, J=6.56 Hz and 2.44 Hz), shown in FIG. 11.

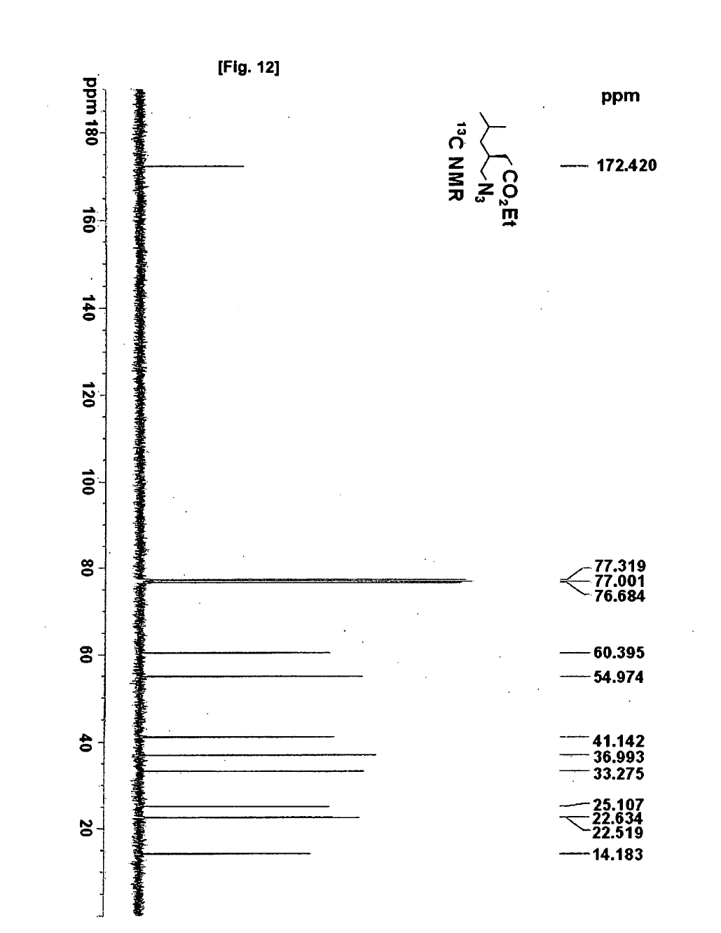

The result of ¹³C NMR (100 MHz, CDCl₃) of the title compound was as follows: δ 172.4, 60.4, 55.0, 41.1, 37.0, 33.3, 25.1, 22.6, 22.5, 14.2, shown in FIG. 12.

The result of HRMS (EI) (C₁₀H₁₉N₃O₂) was as follows: calculated value=213.1477, measured value=213.1475.

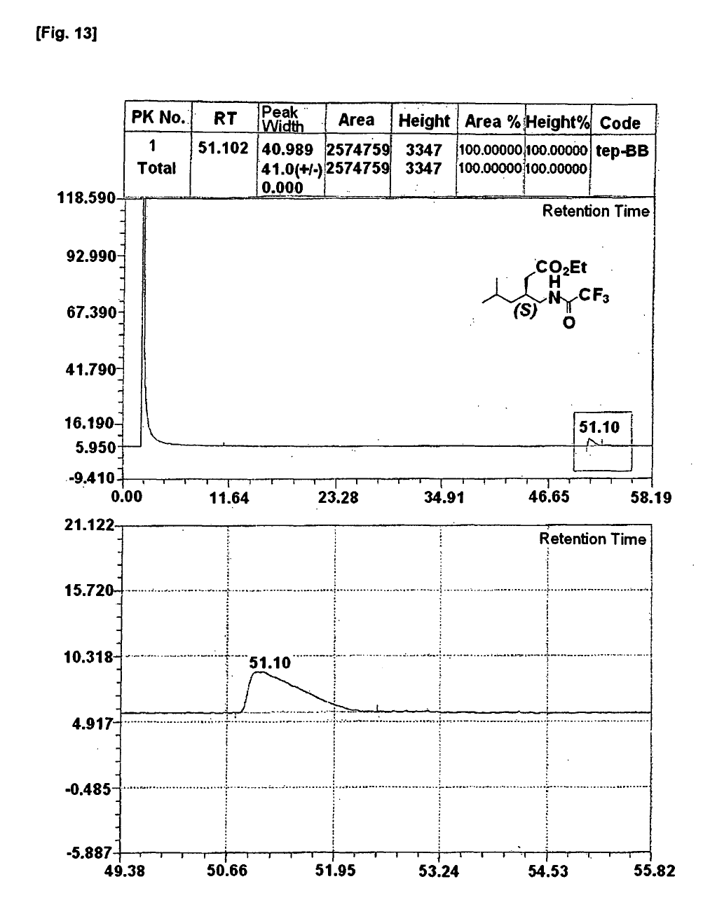

To determine the enantiomeric excess (ee) of the title compound, the compound (10 mg) prepared in this Example, anhydrous trifluoroacetic acid (0.5 mL) and 10% Pd/C (10 mg) were added to ethyl acetate (1 mL), and stirred under a hydrogen balloon for 3 hrs. 10% Pd/C was removed by filtering with cellite, and the filtrate was concentrated under reduced pressure. The concentrated residue was purified by column chromatography using a silica gel (40% ethylacetate/hexane) to obtain a colorless oil. The chiral GC analysis was performed using a chiral DEX β-DM column (130° C. 1.41 kgf/cm^{l , t}ᵣ=51.17). As a result, its enantiomeric excess (ee) was 99% or more. The result of GC analysis is shown in FIG. 13.

### Example 6

### Preparation of Compound 4

The compound ((S)-ethyl 3-(azidomethyl)-5-methylhexanoate, 748 mg, 3.51 mmol) prepared in Example 5 was dissolved in a mixed solvent of THF, MeOH and water (THF:MeOH:water=6:3:1, 30 mL), and then lithium hydroxide monohydrate (736 mg, 17.5 mmol) was added thereto. The reaction mixture was refluxed for 15 min, and the organic solvent was removed under reduced pressure. The aqueous layer was acidified with a 6 N HCl aqueous solution, and then extracted with methylene chloride three times. The combined organic layer was dried over anhydrous sodium sulfate, and concentrated under reduced pressure to obtain Compound 4 ((S)-3-(azidomethyl)-5-methylhexanoic acid) as a colorless oil (590.4 mg, 90.9%).

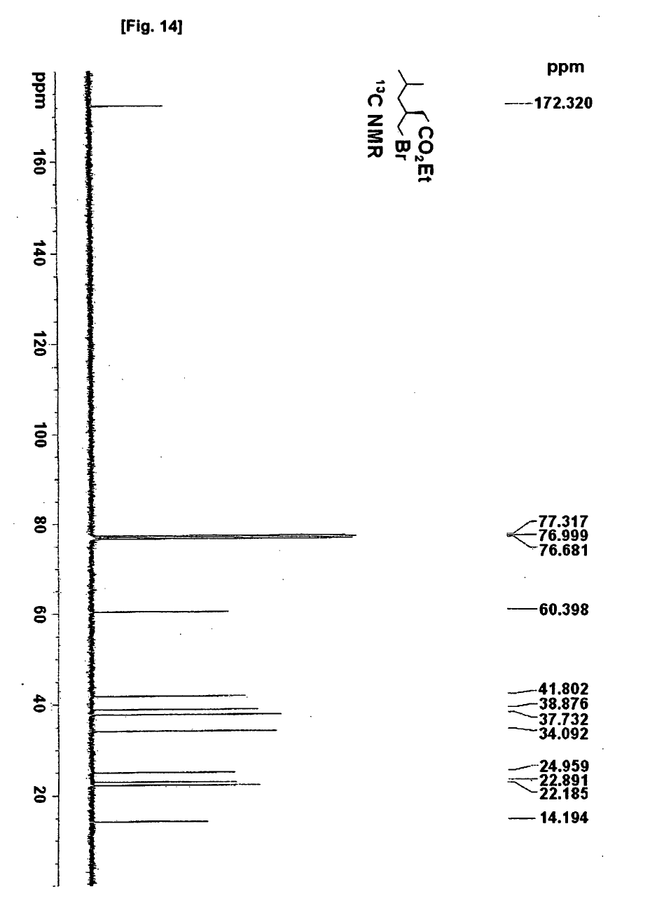

The result of H¹ NMR (400 MHz, CDCl₃) of Compound 4 was as follows: δ 11.24 (1H, br), 3.39 (1H, dd, J=12.24 Hz and 4.92 Hz), 3.29 (1H, dd, J=12.24 Hz and 6.28 Hz), 2.36 (2H, m), 2.15 (1H, m), 1.60 (1H, m), 1.24 (1H, m), 1.18 (1H, m), 0.89 (6H, dd, J=6.58 Hz and 2.20 Hz), shown in FIG. 14.

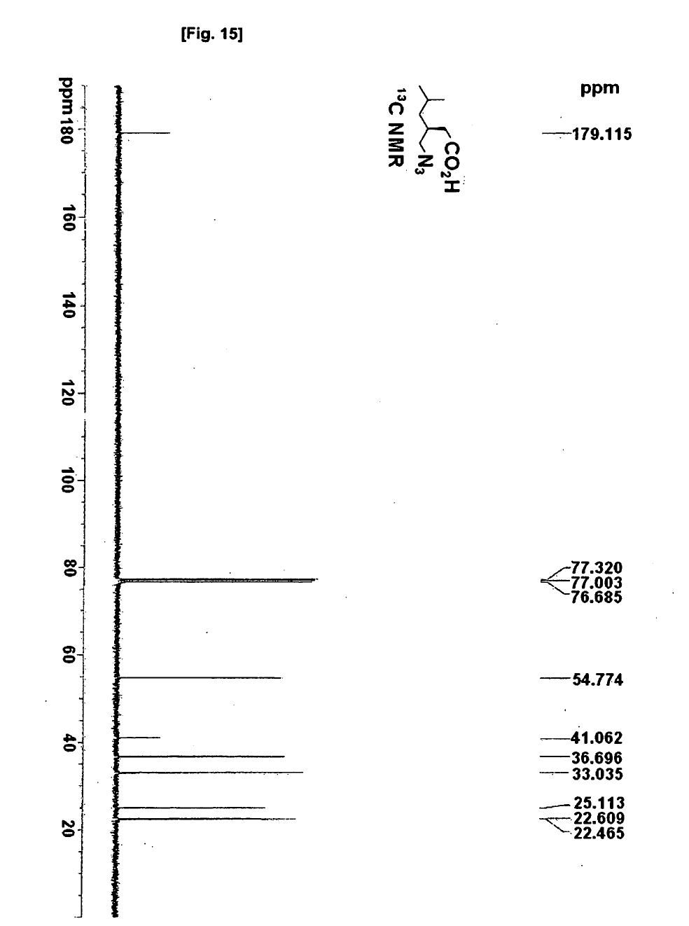

The result of ¹³C NMR (100 MHz, CDCl₃) of Compound 4 was as follows: 6179.1, 54.8, 41.1, 36.7, 33.0, 25.1, 22.6, 22.4, shown in FIG. 15.

The result of HRMS (EI) (C₈H₁₅N₃O₂) was as follows: calculated value=185.1164, measured value=185.1167.

### Example 7

### Preparation of pregabalin (Compound 1)

Compound 4 (569.7 mg, 3.08 mmol) prepared in Example 5 and 10% Pd/C (90 mg) were added to methanol (30 mL), and then stirred under a hydrogen balloon for 3 hrs. 10% Pd/C was removed by filtering with cellite, and the solvent was evaporated under reduced pressure to obtain Compound 1, pregabalin ((S)-3-(aminomethyl)-5-methylhexanoic acid) as a white solid (485 mg, 99.0%).

The obtained solid (Compound 1) had a melting point of 182 to 183° C. and [α]²⁰_{D} was +6.0 (c 0.54, H₂O).

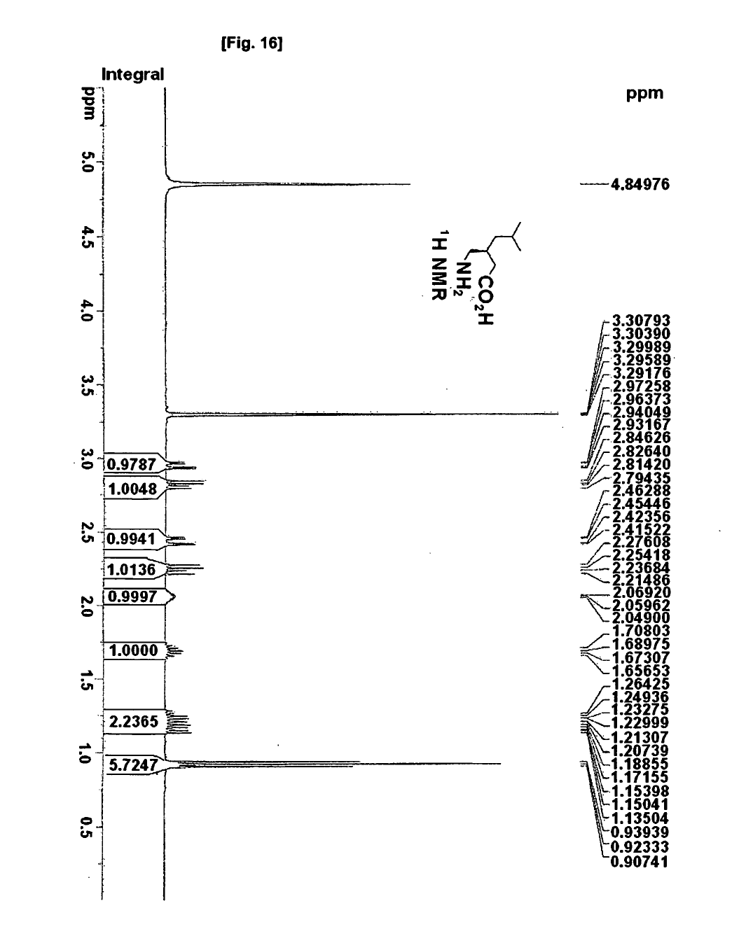

The result of H¹ NMR (400 MHz, CD₃OD) of Compound 1 obtained in this Example was as follows: δ 2.95 (1H, dd, J=12.84 Hz and 3.54 Hz), 2.82 (1H, dd, J=12.82 Hz and 7.94 Hz), 2.44 (1H, dd, J=15.73 Hz and 3.37 Hz), 2.25 (1H, dd, J=15.70 Hz and 8.76 Hz), 2.06 (1H, m), 1.69 (1H, m), 1.23 (2H, m), 0.92 (6H, t, J=6.42 Hz), shown in FIG. 16.

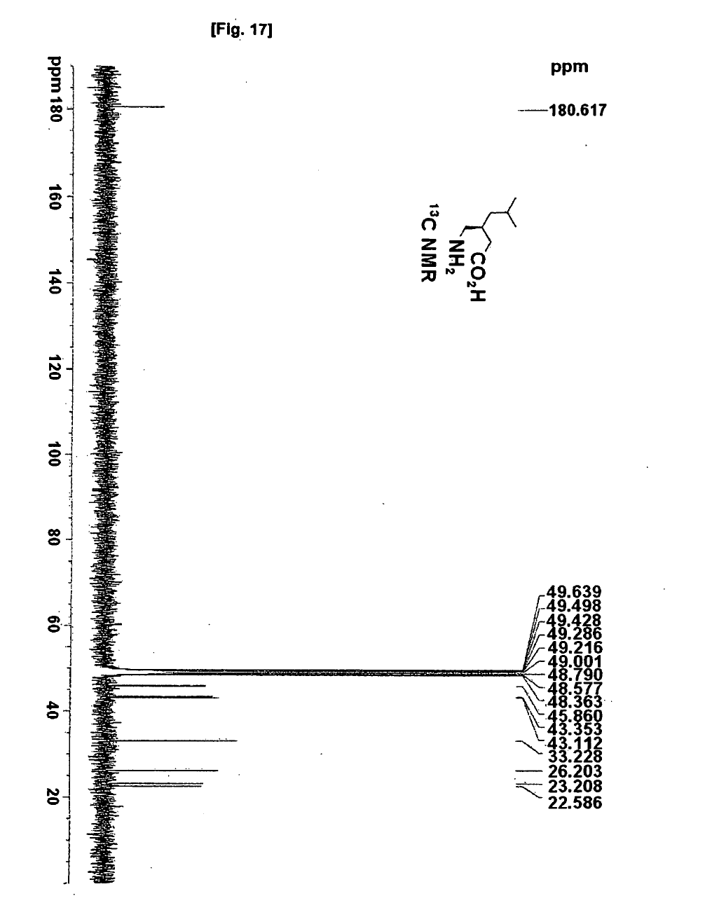

The result of ¹³C NMR (100 MHz, CD₃OD) of Compound 1 was as follows: 6180.6, 45.9, 43.4, 43.1, 33.2, 26.2, 23.2, 22.6, shown in FIG. 17.

The result of HRMS (EI) (C₈H₁₇NO₂) was as follows: calculated value=159.1259, measured value=159.1259.

1. A method for preparing pregabalin of the following Formula 1, comprising the steps of:
1) preparing a lactone compound of the following Formula 3 via cyclopropane ring-opening reaction and decarboxylation of a bicyclic lactone compound of the following Formula 2 by nucleophilic addition of isopropylcuprate;
2) preparing a compound of the following Formula 4 via sequential reactions of halogenation, azidation, and hydrolysis of the lactone compound of Formula 3 obtained in step 1) by lactone ring-opening reaction; and
3) preparing pregabalin of the following Formula 1 by reduction of the compound of Formula 4 obtained in step 2): 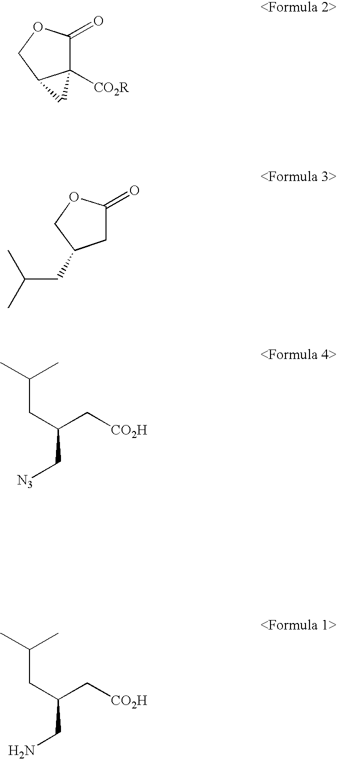
in Formula 2, R is a straight or branched alkyl group having 1 to 6 carbon atoms.

2. The method for preparing pregabalin according to claim 1, wherein isopropylcuprate of step 1) is prepared in-situ in a reactor containing isopropylmagnesium halide represented by Formula i-PrMgX and a copper compound represented by Formula CuY (in Formula i-PrMgX, i-Pr is an isopropyl group, Mg is magnesium, and X is Cl, Br, or I, and in Formula CuY, Cu is copper, and Y is Cl, Br, I, or CN group).

3. The method for preparing pregabalin according to claim 1, wherein the cyclopropane ring-opening reaction and decarboxylation by the nucleophilic addition of step 1) proceed according to the following Reaction Scheme 2 via a compound of Formula 5: 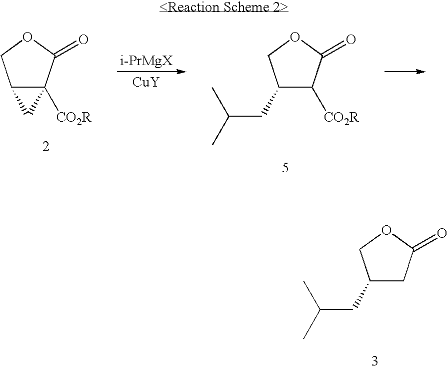 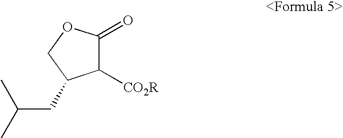
in Reaction Scheme 2, Formula 2, and Formula 5, R is a straight or branched alkyl group having 1 to 6 carbon atoms.

4. The method for preparing pregabalin according to claim 1, wherein in step 2), a compound of Formula 6 is obtained by halogenation, the compound of Formula 6 is subjected to azidation to obtain a compound of Formula 7, and the compound of Formula 7 is subjected to hydrolysis to obtain a compound of Formula 4: 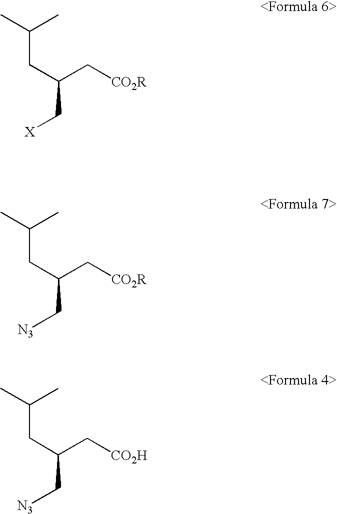
in Formulae 6 and 7, R is a straight or branched alkyl group having 1 to 6 carbon atoms.

5. The method for preparing pregabalin according to claim 4, wherein the halogenation is a step of reacting the compound of Formula 3 with trimethylsilyl halide represented by Formula TMS-X to prepare the compound of Formula 6 (in Formula TMS-X, TMS is trimethylsilyl ((CH₃)₃—Si—), and X is Br or I).

6. The method for preparing pregabalin according to claim 4, wherein the azidation is a step of reacting the compound of Formula 6 with an azide compound represented by Formula MN₃ to prepare the compound of Formula 7 (in Formula MN₃, M is a compound of Group IA including Na and K, and N is nitrogen).

7. The method for preparing pregabalin according to claim 4, wherein the hydrolysis is performed in the presence of a base.

8. The method for preparing pregabalin according to claim 7, wherein the base is selected from alkali metal hydroxide group consisting of lithium hydroxide, sodium hydroxide, and potassium hydroxide.

9. The method for preparing pregabalin according to claim 7, wherein the hydrolysis is performed in alcohol including methanol and ethanol and/or aqueous solvents including tetrahydrofuran (THF) miscible with water.

10. The method for preparing pregabalin according to claim 1, wherein the reduction in step 3) is performed by using a palladium-carbon catalyst.

11. The method for preparing pregabalin according to claim 1, wherein the compound of Formula 2 used in step 1) is prepared by the reaction of (s)-epichlorohydrin of Formula 8 and malonate of Formula 9. 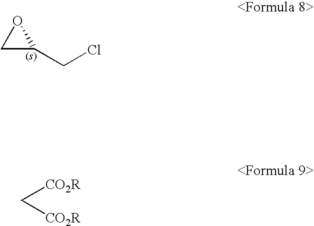
in Formula 9, R is a straight or branched alkyl group having 1 to 6 carbon atoms.

12. The method for preparing pregabalin according to claim 11, wherein the malonate is diethyl malonate.

13. The method for preparing pregabalin according to claim 11, wherein the compound of Formula 2 is a single crystalline form.

14. The method for preparing pregabalin according to claim 11, wherein the compound of Formula 2 has an enantiomeric excess of 99% ee or more.

15. The method for preparing pregabalin according to claim 1, 3, or 4, wherein R is methyl, ethyl, n-propyl, isopropyl or tert-butyl group.
